(19) United States
(12) Patent Application Publication
(10) Pub. No.: US 2025/0040889 A1
(43) Pub. Date: Feb. 6, 2025

(54) CATHETER

(71) Applicant: Auckland UniServices Limited, Auckland (NZ)

(72) Inventors: Bruce Henry Smaill, Auckland (NZ); David Mortimer Budgett, Auckland (NZ)

(21) Appl. No.: 18/917,188

(22) Filed: Oct. 16, 2024

Related U.S. Application Data
(63) Continuation of application No. 17/609,069, filed on Nov. 5, 2021.

(30) Foreign Application Priority Data
May 7, 2019 (NZ) 753258

(51) Int. Cl. A61B 5/00; A61B 5/287; A61B 5/367

(52) U.S. Cl. CPC A61B 5/6859; A61B 5/287; A61B 5/367; A61B 5/6858

(57) ABSTRACT

An open catheter has at least eight splines making up a basket. Each of the splines includes at least six electrodes. An arm is provided connected to and capable of moving the splines from a closed position to an open position, and multiple positions therebetween.

This application is a continuation application of U.S. patent application Ser. No. 17/609,069, filed Nov. 5, 2021, which is a U.S National Stage application under 35 U.S.S. 371 and claims the benefit of priority to International Application No. PCT/IB2020/054254 filed May 7, 2020, which claims the benefit of New Zealand Patent Application No. NZ753258, filed May 7, 2019. The content of U.S. patent application Ser. No. 17/609,069, International Application No. PCT/IB2020/054254, New Zealand Patent Application No. NZ753258 is hereby incorporated by reference herein.

### FIELD OF THE INVENTION

The present invention relates to catheters for use with the determination of physiological information or activation maps of the surfaces of chambers of the heart. In particular, the invention relates to improved open basket catheters.

### BACKGROUND

Electro-anatomic mapping is now widely used to guide treatment of heart rhythm disturbances. This involves the following steps i) 3D heart surface geometry is reconstructed for the chamber (or chambers) of concern ii) electrical signals (time varying surface potentials) are recorded at a number of registered points on the heart surface, and iii) electrical activity throughout the region is rendered, in time and space. Based on this information, likely sources of rhythm disturbance in the heart wall are then located and ablated.

Atrial fibrillation (AF) is the most common heart rhythm disturbance and its prevalence increases with age and heart disease. AF impairs exercise performance, may cause discomfort and increases the risk of stroke. The long-term success of treating persistent and permanent AF with conventional electro-anatomic mapping and ablation methods has been disappointing, see Brooks A G, Stiles M K, et al. Heart Rhythm. 2010;7:835-846.

For example, the widely used CARTO (Biosense Webster, Inc.) mapping system sequentially records electrical activity and 3D coordinates at individual points across the endocardial surface of a heart chamber. This enables reliable electro-anatomic maps to be reconstructed when electrical activity is repetitive, but not in persistent or permanent AF when spatio-temporal electrical activity is highly variable.

This has driven recent development of methods for near real-time mapping and analysis of electrical activity in persistent and permanent AF using intracardiac catheters that record electrical activity simultaneously at multiple 3D locations. In this setting, real-time covers acquisition, analysis and visualization processes that are completed within a few seconds at most.

One approach here is to use flexible multi-electrode basket catheters that make direct contact with the atrial surface. Electrical activity can be mapped throughout the cardiac cycle provided that electrodes remain in contact with the chamber wall and their 3D position is known.

The Constellation catheter (Boston Scientific, Inc.) is an expandable basket catheter with 64 electrodes to record potentials. Constellation catheters in a contact mapping system have detected rotors (or focal drivers) in patients with AF for the first time and almost doubled the success rate of catheter ablations by targeting rotor circuits directly [Narayan S M, Krummen D E, et al. JACC. 2012;60:628-636-846]. This has led to the development of improved catheter design and phase mapping software by Topera Medical.

However, such contact mapping approaches have a number of inherent limitations. For successful real-time mapping across a complete atrial chamber, catheter dimensions need to be matched to those of the chamber of interest. Even if this can be done, the complexity of atrial anatomy means that some regions cannot be mapped adequately. Basket catheters with dimensions appropriate for global atrial mapping cannot easily be introduced into the atrial appendages or into the junctions of the pulmonary veins. Furthermore, a significant number of electrodes will not make good contact with the chamber wall throughout the cardiac cycle, which further limits anatomic resolution. Finally, the presence of a large basket catheter in an atrial chamber constrains deployment and positioning of other devices such as ablation catheters.

An alternate approach is to use noncontact mapping methods. Here, electrical activity is measured on a surface adjacent to the inner or outer surface of the cardiac chamber of interest and is then mapped onto the heart surface in question using inverse problem techniques. St Jude Medical markets a catheter and mapping system intended for noncontact 3D electro-anatomic mapping. The catheter consists of a 64-electrode array mounted on an inflatable balloon, but this device is not widely used for mapping AF. Reasons for this are that the closed balloon partially occludes the atrial chamber. Also, the electrodes on the balloon are often too far from the atrial wall for accurate reconstruction of surface activation (atrial dilatation is common in longstanding persistent AF).

Acutus Medical is developing a complete mapping system based on an expandable basket catheter that contains 42 electrodes as well as ultrasound probes. With this approach, electrical activity recorded with a multi-electrode basket catheter in an atrial cavity is used to estimate an equivalent electrical dipole distribution within the atrial wall. A weakness of this approach is that the distribution is an inferred measure that cannot be equated directly with the surface potentials measured by clinicians during the ablation process. Furthermore, the low channel count constrains the spatial resolution that can be achieved, and the dimensions of the catheter preclude its use in the atrial appendages or pulmonary vein junctions.

CardioInsight maps electrical activity measured on the body surface with a multi-electrode vest onto the epicardial surface of the heart using a well-established inverse method. The approach is non-invasive, but it requires accurate 3D anatomic representations of body surface and epicardial geometry using computed tomography (CT) or magnetic resonance imaging (MRI). Weaknesses include the lack of spatial resolution in mapping atrial electrical activity and the fact that the epicardial electrical activity reconstructed with this approach cannot be directly related to the endocardial activity recorded by clinicians during AF ablation.

The Ensite multi-electrode array catheter is a closed catheter with dimensions of 18×46 mm, which can restrict ablation catheter manipulation. The reconstructed activation patterns can be inaccurate if the distance from the mapped area to the centre of the multielectrode array is more than 40 mm, common where atria are dilated.

U.S. Pat. No. 7,505,810 describes a non-contact cardiac mapping system including pre-processing. The system focusses on solving the inverse problem for a catheter in the heart by pre-processing matrices to speed performance. The system solves the inverse problem in the space between the endocardial surface and a closed catheter surface where there is no surface flow.

### OBJECTS OF THE INVENTION

It is an object of the invention to provide an improved open basket catheter to assist in determining physiological information of an endocardial surface which will at least go some way to overcoming disadvantages of existing catheters or systems, or which will at least provide a useful alternative to existing systems.

Further objects of the invention will become apparent from the following description.

### SUMMARY OF INVENTION

Accordingly in one aspect the invention may broadly be said to consist in an open catheter comprising:
at least eight splines making up a basket,
each of the splines includes at least six electrodes,
an arm connected to and capable of moving the splines from a closed position to an open position, and multiple positions therebetween.

Preferably the electrodes on the splines provide an array of electrodes.

Preferably the electrode array may be altered by withdrawing or advancing the splines into or out of the arm of the catheter.

Preferably the basket is steerable.

Preferably the electrode array and thus splines can be locked into any one of a multitude of dimensions between fully open and fully closed states.

Preferably the electrodes are uniformly spaced as far as is possible in open and closed states and distributed evenly across the mathematically closed virtual surface that bounds them.

Preferably the splines are flexible and make up a flexible basket.

Preferably the splines are made from flexible printed circuit boards. Preferably each of said electrodes is evenly distributed along each of said splines.

Preferably said even distribution is a uniform distribution.

Preferably said distribution is a dense electrode distribution.

Preferably the electrode array is arranged so as to provide substantially even coverage over the catheter surface.

Preferably the electrodes are uniformly distributed in all splines such that the neighbouring electrodes have the least linear distance from each other.

Alternatively the electrode distribution can be changed by expanding or contracting the basket to maximise resolution of data recorded by the electrodes.

Preferably the splines are adjustable by being withdrawn or advanced out of the arm of the catheter. Preferably the electrodes are non-contact in use.

Preferably the catheter arm has markings to indicate the advancement of the splines.

Preferably the catheter arm has markings to indicate the expansion or contraction of the basket.

Alternatively, the catheter arm includes wheel indicating the amount of advancement of the splines.

Preferably the catheter includes an ablation device at the end of the catheter, preferably extending out from the basket.

Alternatively, the splines are an array of splines where some of the splines have more electrodes distributed thereon than others.

Preferably the catheter has 16 splines making up the basket.

Preferably the splines include at least 6 electrodes.

Accordingly, in a second aspect the invention may broadly be said to consist in an open catheter comprising:
an array of splines making up a basket,
each of the splines including a multitude of electrodes and each of said multitude of electrodes are evenly distributed along each of said splines.

an arm connected to and capable of moving the splines from a closed position to an open position, and multiple positions therebetween.

Preferably said array of splines is made up of eight splines. Preferably each of the splines includes at least six electrodes.

Preferably the electrode array may be altered by withdrawing or advancing the splines into or out of the arm of the catheter.

Preferably the basket is steerable.

Preferably the electrode array and thus splines can be locked into any one of a multitude of dimensions between fully open and fully closed states.

Preferably the electrodes are uniformly spaced as far as is possible in open and closed states and distributed evenly across the mathematically closed virtual surface that bounds them.

Preferably the splines are flexible and make up a flexible basket. Preferably the splines are made from flexible printed circuit boards.

Preferably said even distribution is a uniform distribution.

Preferably said distribution is a dense electrode distribution.

Preferably the electrode array is arranged so as to provide substantially even coverage over the catheter surface.

Preferably the electrodes are uniformly distributed in all splines such that the neighbouring electrodes have the least linear distance from each other.

Alternatively the electrode distribution can be changed by expanding or contracting the basket to maximise resolution of data recorded by the electrodes.

Preferably the splines are adjustable by being withdrawn or advanced out of the arm of the catheter. Preferably the electrodes are non-contact in use.

Preferably the catheter arm has markings to indicate the advancement of the splines.

Preferably the catheter arm has markings to indicate the expansion or contraction of the basket.

Alternatively, the catheter arm includes wheel indicating the amount of advancement of the splines.

Preferably the catheter includes an ablation device at the end of the catheter, preferably extending out from the basket.

Alternatively, the splines are an array of splines where some of the splines have more electrodes distributed thereon than others.

Alternatively the catheter has 16 splines making up the basket.

Preferably the splines include at least 6 electrodes.

Accordingly in a further aspect the invention may broadly be said to consist in a system for determining the physiological information of an endocardial surface the system comprising:

a catheter adapted to be inserted into an endocardial chamber, the catheter having a plurality of electrodes adapted to measure physiological information, a processing means for receiving information from the plurality of electrodes and processing the information into physiological information of the electric field at the catheter surface.

a processing means for receiving the information of the electric field at the catheter surface and processing the information into physiological information of the physiological information of the endocardial surface.

Preferably the system comprises a means of calculating the position of the catheter.

Preferably the position is relative to the endocardial surface.

Preferably the system comprises a means of generating a representation of the endocardial surface.

Preferably a processing means receives the position of the catheter and processes the position of the catheter surface relative to the endocardial surface.

The disclosed subject matter also provides method or system which may broadly be said to consist in the parts, elements and features referred to or indicated in this specification, individually or collectively, in any or all combinations of two or more of those parts, elements or features. Where specific integers are mentioned in this specification which have known equivalents in the art to which the invention relates, such known equivalents are deemed to be incorporated in the specification.

Further aspects of the invention, which should be considered in all its novel aspects, will become apparent from the following description.

### DRAWING DESCRIPTION

A number of embodiments of the invention will now be described by way of example with reference to the following.

FIG. 1(a) and FIG. 1(b) are schematic representations of prior art catheters where FIG. 1(a) shows an open catheter with electrodes spaced along splines and FIG. 1(b) shows the closed virtual surface defined by the electrodes. The electrodes are electrically connected via conductors through a flexible tube to the proximal end of the catheter where it is connected to additional recording equipment (not shown).

FIG. 2(a) is a schematic representation of a system embodiment showing a catheter in the left atrium and FIG. 2(b) is a schematic representation of an atrial electrogram from one electrode.

FIG. 3 shows a schematic diagram of a catheter in a heart and additional recording, control and processing devices that are required for inverse endocardial mapping.

FIG. 4(a) and FIG. 4(b) are schematic diagram of an expandable catheter of the present invention, shown in the open state for global panoramic mapping in FIG. 4(a) and in the semi-closed state for region-of-interest mapping in FIG. 4(b).

FIG. 5 shows an illustration of a multifunctional catheter of the present invention.

FIG. 6 is an illustration of another embodiment of a catheter of the present invention that includes distance markers on the cable systems.

FIG. 7 is an illustration of a guiding catheter hand piece, including a thumb wheel that causes the basket catheter to expand.

FIG. 8 is an illustration of yet another embodiment of a catheter of the present invention that has a greater distribution of electrodes on some spines compared to other splines.

FIG. 9 shows a distribution of 64 points on spherical surface, the points being generated from MATLABO. This shows that the linear spacing between neighbouring points can be iterated until all are approximately equally spaced. The rough estimate of space in between neighbouring points is ˜9.6 mm. This configuration shown an optimal electrode distribution to achieve uniform coverage for measuring the electrical potential distribution. A physical catheter will have constrains on how close the electrodes can be positioned to these ideal locations.

FIG. 10 shows catheter designs with electrodes assemblies for increasing spline numbers, a) 10 splines, b) 12 splines, c) 14 splines, d) 16 splines and e) 18 splines.

FIG. 11 shows various mechanical parts of an alternative embodiment to locate the splines in their correct positions of the catheter of the present invention.

FIG. 12 shows a comparison between a prior art catheter and the catheter of FIG. 11.

FIG. 13 shows an embodiment of a spline of the catheter of the present invention being a flexible circuit board containing electrodes. In this embodiment electrodes and conductors are located on multiple layers of the circuit board.

FIG. 14 shows the PCB layouts for connecting the splines of FIG. 13 to the UnEmap system.

FIG. 15 is an illustration of an embodiment of the catheter of the present invention where the open basket catheter is made of 16 splines with 6 electrodes each.

FIG. 16(a) and FIG. 16(b) illustrate a prototype version of the catheter of FIG. 15.

FIG. 17 is an illustration of a saline bath setup used for testing the prototype catheter of FIG. 16.

FIG. 18 shows illustrations of the importance of the electrode locations on the splines of a catheter and shows the methods for assessing the performance of one catheter design against a different catheter design.

### DETAILED DESCRIPTION OF THE DRAWINGS

Throughout the description like reference numerals will be used to refer to like features in different embodiments.

An open multi-electrode catheter of the present invention may be used with a mapping system that is capable of reconstructing panoramic electrical activation in atrial chambers simultaneously by intracavity inverse mapping. A mapping system that may be used with the catheter of the present invention is described in US patent U.S. Pat. No. 10,610,112 the contents of which are included herein.

The mapping system disclosed in U.S. Pat. No. 10,610,112 provides a means of reconstructing panoramic electrical activity in a heart chamber from physiological information, most particularly, time-varying electrical potentials (may also be referred to as electrical fields or fields) recorded using an open catheter inside the chamber that contains multiple electrodes, some or all which are not in contact with the wall of the chamber. A numerical approach is used to estimate physiological information (most preferably electrical potentials, electrical fields or fields) in the volume bounded by the electrodes on the catheter from the recorded potentials. This provides the additional boundary conditions necessary for accurate inverse mapping of potentials onto the inner surface of the heart chamber. For instance, in inverse solution packages that employ Boundary Element Methods (BEMs), it is necessary to specify both potential and potential gradients at measurement points.

The mapping system enables rapid reconstruction and visualisation of electrical potentials on the endocardial surface of a cardiac chamber or region of that chamber preferably from electrical potentials measured with an expandable multi-electrode basket catheter, in which either all or some of the electrodes are not in contact with the surface. Such a catheter is open in a sense that blood within the chamber passes freely through it, but in which the electrodes define a mathematically closed 3D surface.

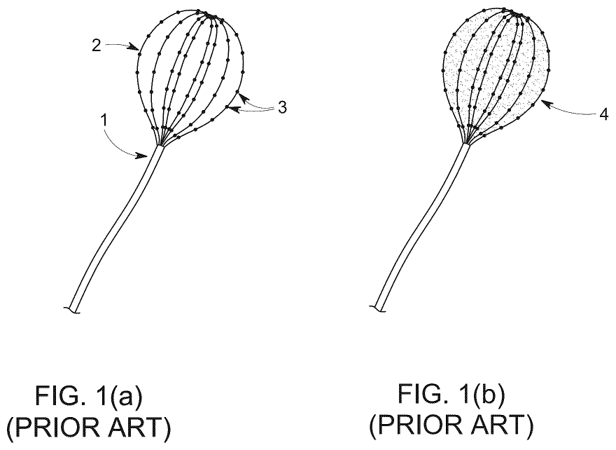

FIG. 1 shows a schematic representation of a multi-electrode mapping catheter 1 of the prior art. It consists of multiple expandable splines 2 with electrodes 3 spaced along the splines. The catheter is open in the sense that fluid can pass freely between the splines. However, as shown in FIG. 1b, all electrodes lie on a continuous virtual surface 4 that is closed in the mathematical sense.

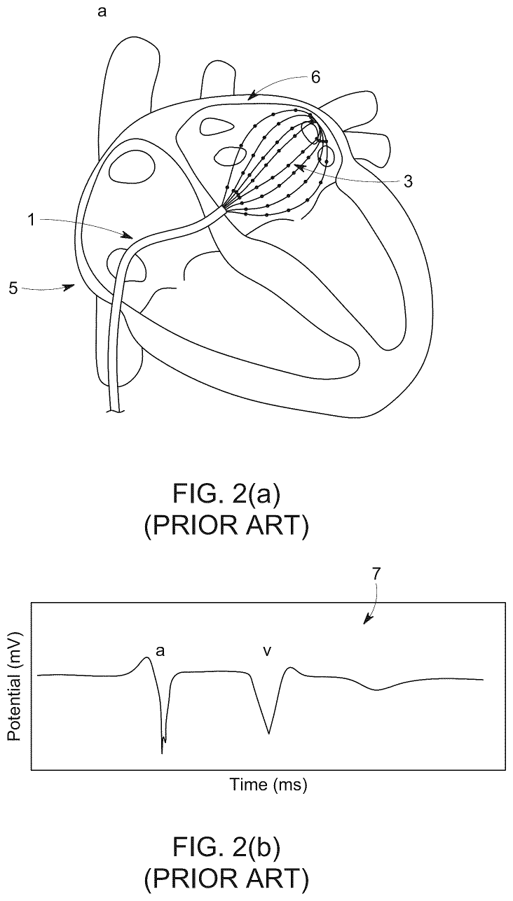

FIG. 2a shows a schematic representation of the mapping problem in a heart 5. A catheter 1 may be located in the left atrium (LA), and electrical potentials generated by electrical activity in the heart can be recorded by each of the multiple electrodes simultaneously. An electrogram 7 (potential as a function of time) at a typical electrode 3 is displayed for a single cardiac cycle in FIG. 2b. The potential distribution on the LA endocardial surface 6 at successive instants through the cardiac cycle must be reconstructed based on the corresponding potentials recorded at the multiple catheter electrodes. This typically involves an inverse approach or solving an inverse problem. The objective of the inverse problem is to reconstruct source information (e.g. atrial endocardial potentials) from the measured field (e.g. potentials recorded at the catheter electrodes) based on a priori information on the physical relationships between sources and measured field. In this setting, information is also required about the 3D geometry of the endocardial surface and the 3D location of each of the electrodes.

FIG. 2a shows the four cardiac chambers: the left atrium (LA), right atrium (RA), right ventricle (RV) and left ventricle (LV). An endocardial surface 6 is typically the surface of one of the chambers of the heart. Where discussed herein the endocardial surface may be represented as a 2D surface, but it is understood that a user of the system would typically be investigating a 3D endocardial surface enclosing a chamber within. In some embodiments an endocardial surface may be only a portion of a chamber, that portion being of interest.

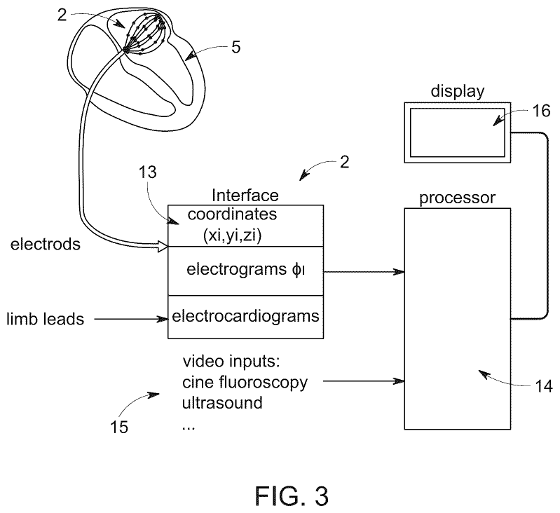

FIG. 3 shows a diagram of the mapping system of U.S. Pat. No. 10,610,112 in use. A catheter is placed inside a volume of interest, typically a heart chamber. Catheters are electrically connected to an interface 13, which is electrically isolated and may comprise a proprietary system or a set of such systems. Instantaneous potentials and the 3D positions are acquired from individual electrodes on one or more cardiac catheters. For instance, potentials and 3D positions may be recorded simultaneously from multi-electrode basket catheters positioned in the RA and LA, or from a multi-electrode basket catheter and an ablation catheter in the same cardiac chamber. 3D electrode positions are recorded using impedance techniques, magnetic sensors, ultrasound sensors or combinations of these methods.

Electrocardiograms (ECGs) are also acquired without position information for standard lead configurations.

The processing unit 14 controls the acquisition and processing of data so that recorded potentials or information derived from them can be mapped onto the endocardial surface of a heart chamber or chambers in a form that is useful to the operator.

The first processing step is to construct a computer representation of the 3D endocardial surface geometry of the heart chamber or chambers of interest. This may be derived from i) cardiac MR images ii) contrast-enhanced cardiac CT images or iii) surface coordinates mapped under fluoroscopic guidance using a catheter. Alternately, geometry created in iii) can be merged with endocardial surfaces segmented from i) or ii). Preferably, static 3D models will be integrated with cine-fluoroscopic imaging or ultrasound imaging to provide estimates of heart wall motion. Provision for the import of such video data is indicated as 15. Endocardial potentials will be rendered on a computer representation of the 3D surface of the heart chamber or chambers presented on a screen or display device 16 in a form that can be manipulated interactively by the operator. The location of catheter or catheters with respect to the heart wall will also be displayed.

As discussed above, multi-electrode catheters are currently inserted into the heart atria to map the electrical activation within the heart and to help with guiding ablation to treat atrial fibrillation. Current catheters rely on contacting the internal wall of the atria to obtain useful electrical information, their design is orientated to achieving electrode contact. With the mapping system of U.S. Pat. No. 10,610,112 and the possibility of using non-contact catheters, catheters can be designed to provide best coverage of the atrial endocardium.

In a first embodiment the multi-electrode catheter of the present invention has an electrode distribution that can be changed, not to maximise contact, but to maximise the resolution of the atrial electrical activation data.

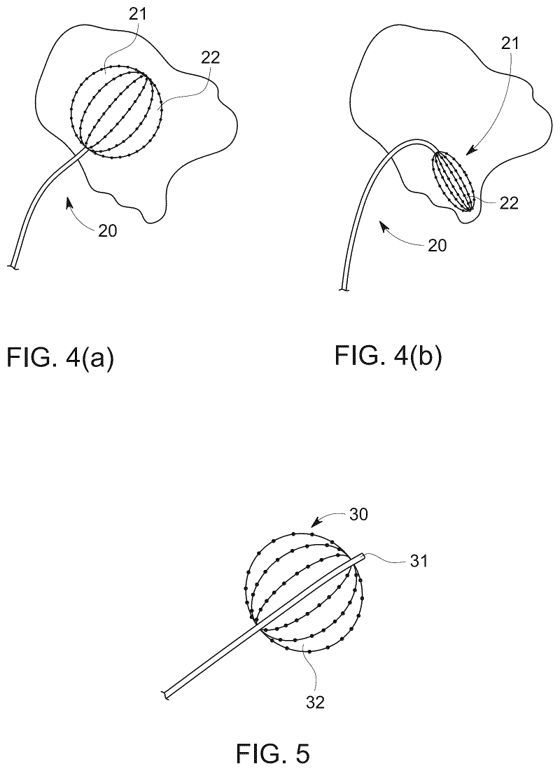

FIG. 4 shows a method of operating a catheter of the present invention in a sequence of steps guided by the information displayed 16 from a system as described in relation to FIG. 3. Initially a global picture of electrical activity on the endocardial surface of a heart chamber may be acquired and displayed. Preferably this will use a catheter 20 with a basket 21 positioned centrally with electrodes 22 in contact with or adjacent to as much of the endocardial surface of the heart chamber as possible. FIG. 4a shows a catheter 20 being used for global mapping. FIG. 4b shows how the catheter 20 with smaller dimensions (as adjusted by a user) may be used to map in specific regions of the chamber with greater precision, because it can be moved close to the endocardial surface. So, after obtaining the data to produce a global map of electrical activation, the catheter basket 21 can be made smaller and can then be manoeuvred to locate the more compact electrode 22 set nearer to an atrial wall of greater interest.

In a preferred method of the mapping system, global mapping of electrical activity is obtained over a short period of time (for instance continuous periods of at least 10-20 seconds are required in AF) before a user decides which areas require further investigation. Higher resolution mappings will be obtained in these regions-of-interest by moving multi-electrode arrayed catheters with smaller diameters into them (again in AF continuous periods of at least 10 to 20 seconds are required for region-of-interest mapping). This method will support more efficient high-resolution endocardial mapping of electrical activity because it utilizes potentials recorded at all electrodes whether they are in contact with the endocardial surface of the heart chamber or not. The operator will also receive direct feedback on the accuracy of endocardial maps through visual comparison of maps and electrograms displayed as the catheter is moved closer to the surface and as some electrodes make contact with it.

The mapping approach above could be carried out using combinations of catheters with different dimensions. However, in preferred forms of the invention, a single adjustable catheter may be used. With such a single adjustable catheter, the dimensions of the electrode array may be altered by withdrawing or advancing the splines into or out of the catheter. Preferably the catheter is steerable. Preferably it will be possible to lock the dimensions of the electrode array into any one of a multitude of dimensions between fully open and fully closed states. Preferably the electrodes are uniformly spaced as far as is possible in open and closed states and distributed evenly across the mathematically closed virtual surface that bounds them. Preferably inter-electrode spacing will be sufficient to characterize electrical activity appropriately within endocardial regions on the order of 10 mm in diameter.

Preferably it will be possible to introduce the catheter into atrial appendages and pulmonary veins in a closed state.

Another embodiment of a catheter 30 of the present invention is to place a basket of electrodes 32 around the head of an ablation catheter 31 to form a multi-functional catheter (see FIG. 5). Thus, global measurements may be obtained with a conventional basket catheter, then the multi-functional catheter of the present invention may be inserted, and regional searches may be performed. When a candidate ablation site is determined, local (or regional) mapping can be performed immediately prior and after ablation without the need for changing catheters. This catheter provides real-time electrical mapping feedback while the ablation tip is still in the atria and available for further ablations.

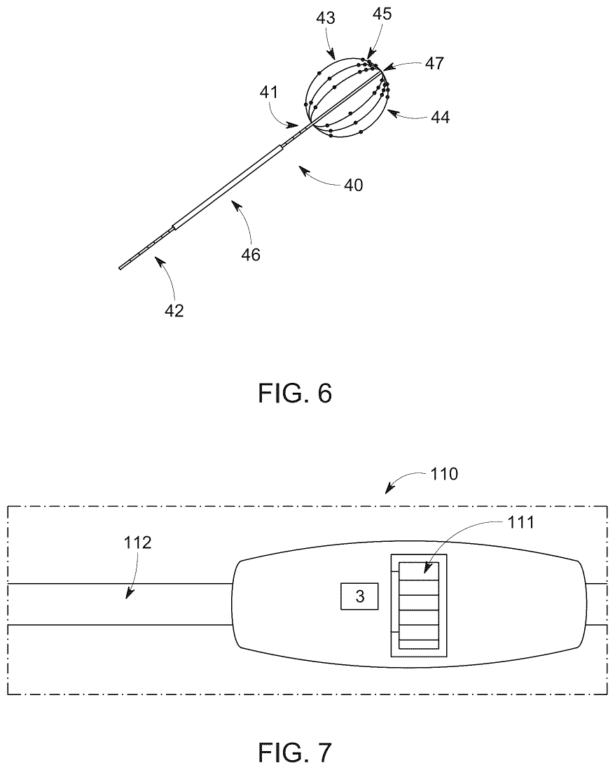

Another embodiment of a catheter 40 of the present invention, see FIG. 6, includes distance markers 41, 42 on the cable systems which puts the splines 43 into compression and causes the basket catheter 44 to expand in size. These markers allow the extension to be precisely known such that the distribution of the catheter electrodes 45 with respect to each other is known. This simplifies (and speeds up) the computational process for calculating electrical activation patterns. Markers on the tensioning cable and on the sheath, both contribute to knowing the shape of the catheter basket 44 and electrode 45 positions.

Alternatively, in various embodiments of the catheter of the present invention, as described, when in use, fluoroscopic imaging may be used to visualise the catheter and give confidence to the user that it is deployed correctly.

FIG. 7 shows a mechanism 110 that can be used with a catheter to enable guiding of the catheter. This may be for use with any of the basket catheters herein described. The mechanism 110 includes a wheel 111 in the hand or arm piece 112, where the turning of the wheel 111 extends the cable system and controls the catheter expansion. The position of the wheel indicates the extent of the catheter expansion. For example, in FIG. 7, the indicator currently reads “3”—which represents a 30% extension.

Yet another embodiment of the catheter of the present invention distributes more electrodes 45 at the distal end of the catheter splines and less electrodes at the proximal end, see example illustration in FIG. 6. When the catheter 40 is at a smaller size, some of the proximal electrodes are withdrawn into the catheter sheath 46, but the higher density electrodes are still blood/body contacting at the distal end 47.

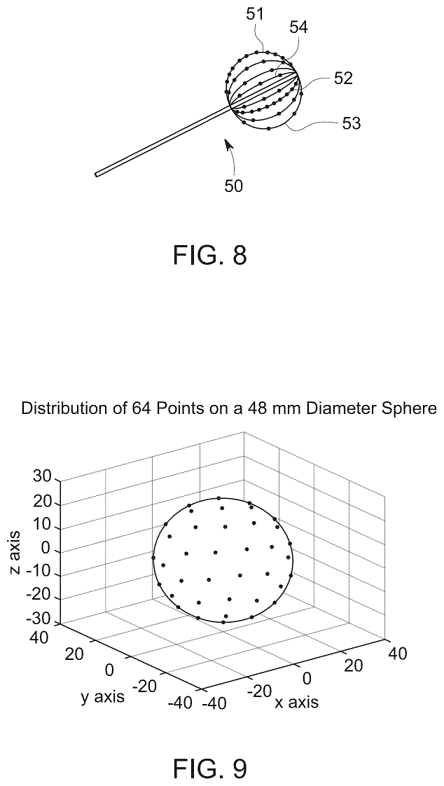

Yet another embodiment of a catheter 50 of the present invention, the catheter 50 may have a greater distribution of electrodes (see splines 51, 52 in FIG. 8) on some spines compared to other splines (see splines 53, 54 as examples). This catheter does not have axial symmetry and as such more electrodes can be orientated towards a specific atrial wall through rotation of the catheter. This is helpful when doing regional mapping because a greater number of electrodes can be positioned close to the atria wall in the area of interest.

Any of the catheters described above, or indeed below, may be used in a method for defining the size of the catheter basket. A procedure according to such methods is to insert a catheter fully contained within a sheath and then expand the catheter basket once located in the atria. Signal processing of the data from each electrode on the splines of the basket will show when an electrode makes contact with the atrial wall. The basket can continue to be expanded until electrodes at, at least one other different location is identified as experiencing wall contact. At this size, the electrical signals from the basket will be subject to motion artefact as the heart beats. The size of the basket can then be reduced to prevent multi-electrode wall contact on a beat by beat basis. This process is optimised to produce the largest basket size (placing electrode close to the atria wall) without inducing motion artefact.

Note, the number of splines as shown on the catheters in FIGS. 4 to 8 are for illustration and explanation purposes only. Catheters of the present invention may have more or less splines dependent on requirements.

### Catheter Design Process

To improve the current methods for the electrical mapping of the atrial endocardial surface new multi-electrode catheters (such as those described above, and additionally below) are needed. A multi-electrode basket catheter must provide good coverage for the region of interest based on non-contacting electrodes. It should easily be expanded to fill the atria or contracted to support high-density electrode mapping in a smaller ROI. The inventors have discovered that good coverage of the atria can be achieved when the electrodes on a catheter are uniformly distributed over the catheter surface. In addition, as the catheter basket is open blood within the atria is allowed to flow. We discuss below some design considerations for optimal placement of electrodes on a catheter spline assembly. We have also attempted to determine how many electrodes and splines would yield more accurate endocardial maps.

The initial design process that the inventors conducted involved distributing 64 electrodes uniformly over a 48 mm diameter spherical surface. The number of electrodes and sphere diameter are based on the parameters of a ConstellationTM catheter (Boston Scientific) which is the most widely used catheter. Using MATLAB© (The Mathworks, Natick, Massachusetts), the smallest spacing between the distributed 64 points was determined. A uniform distribution is achieved when the straight-line distance between neighbouring points is the same. Calculations showed that this electrode spacing could be as low as 9.6 mm. FIG. 9 shows the uniform distribution of 64 points on a sphere generated in MATLAB©.

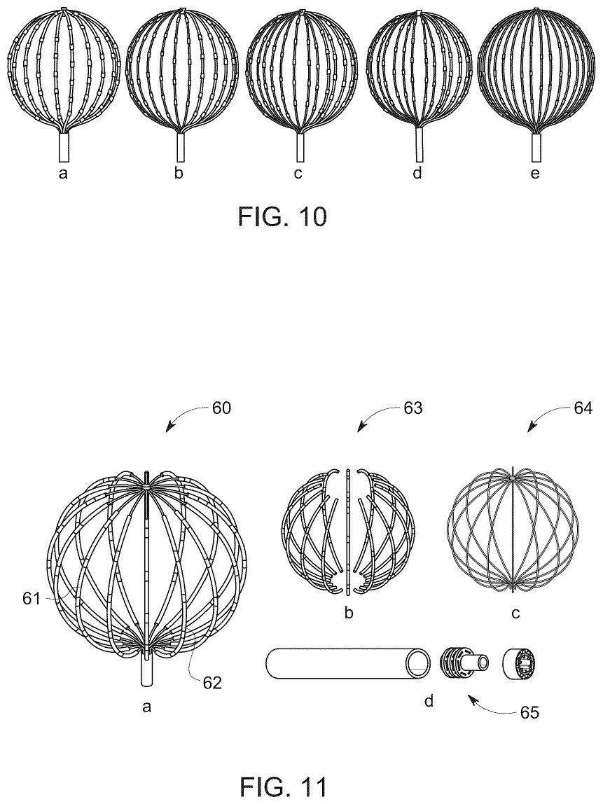

Different open basket catheter designs were then created in Solidworks™ to visualise the assembly and distribution of 64 electrodes when confined to being located on 10, 12, 14, 16 or 18 splines. Another design constraint was added which required packing the splines into the space available inside an 8.5 Fr (˜2.83 mm) diameter catheter sheath. FIG. 10 shows the five different catheter assemblies created in Solidworks™, labelled a to e. The number of electrodes per spline is not equal. The design brief was to distribute the 64 electrodes over the splines such that neighbouring electrodes have the least linear distance from each other. All basket assemblies have the following similar dimensions:
a) diameter of 48 mm,
b) spline diameter of 0.6 mm, and
c) electrode length of 3 mm.

Thus, design output produces a different number of electrodes per spline for each basket configuration.

The five catheter designs were then compared with respect to their spline spacing and average electrode distance. Table 1 shows the comparison made for the designs. As expected, catheters with more splines were able to reduce the inter-electrode distance. Analysis of the packing density showed that the 16-spline catheter would still fit inside an 8.5 Fr catheter sheath. The analysis was done by calculating the total number of 0.6 mm diameter spline that could be packed in a 2.83 mm diameter sheath.

TABLE 1
Comparison of Multi-Electrode Basket Catheter (MBC) Models

| | Maximum | Minimum | Maximum | |
|---|---|---|---|---|
| | Spacing | Number of | Number of | Average |
| Number of | Between | Electrodes/ | Electrodes/ | Electrode |
| Splines | Splines (mm) | Spline | Spline | Distance (mm) |
| 10 | 15.12 | 6 | 7 | 17.57 |
| 12 | 12.58 | 5 | 6 | 12.55 |
| 14 | 10.09 | 4 | 6 | 11.8 |
| 16 | 9.45 | 3 | 5 | 10.78 |
| 18 | 8.41 | 3 | 4 | 10.63 |

The 16-spline catheter design was further improved by using an equal number of electrodes per spline. A SolidWorks™ render of an improved 16-spline catheter is shown in FIG. 11. The full catheter is made of the following; a. basket assembly 60 for the 16 splines 61 with 4 electrodes 62 per spline, b. spline cover/sleeves 63 containing the electrode details, c. a nitinol frame 64, which provides shape and flexibility, and d. catheter body 65 with locking mechanism holding the parts together.

The spline cover/sleeves are preferably slidable and biocompatible. In preferred forms they may be made of polyurethane or polyimide. They preferably have an outer diameter of 1 mm and 0.025 mm wall thickness. The electrodes are preferably made of platinum-iridium rings, preferably having a length of 1.27 mm and a 1 mm outer diameter. The sleeves preferably cover the frame and copper signal wires. Nitinol is an alloy of nickel and titanium that has a shape memory property. Preferably, the frame has a rectangular cross-section with dimensions of 0.2 mm by 0.4 mm. The frame preferably has a diameter of 48 mm. The catheter body holds the catheter together and is comprised of a locking mechanism to fix together the sleeves and the frame. The locking mechanism 65 is preferably a locking ring and anchor, preferably both made of titanium, however other appropriate locking mechanisms and materials may be used. The locking mechanism is preferably tubular in order for copper wires connected to the electrodes to run through it.

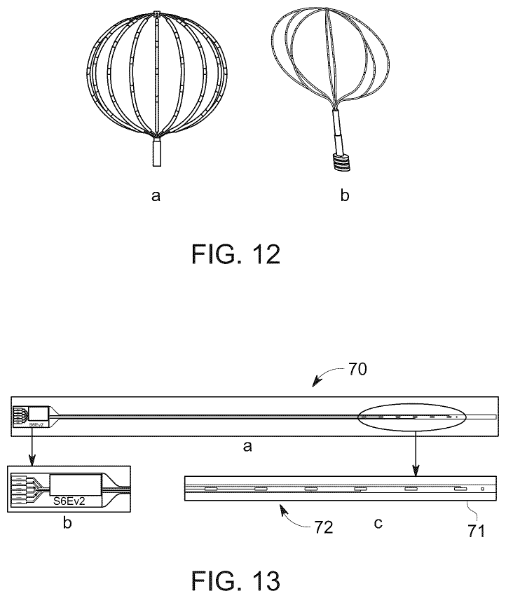

The improvement in catheter surface coverages is illustrated in FIG. 12 where the 16-spline catheter (a) is able to locate electrodes with a maximum distance of 9.45 mm, in the ConstellationTM catheter (b) the distance between the electrodes along a spline is much smaller, but between spines is much greater (maximum at the equator). The additional splines improve the distribution of electrodes compared to existing catheters. The catheter of this embodiment provides a denser electrode distribution than prior art catheters that may help provide good coverage for region-of-interest mapping.

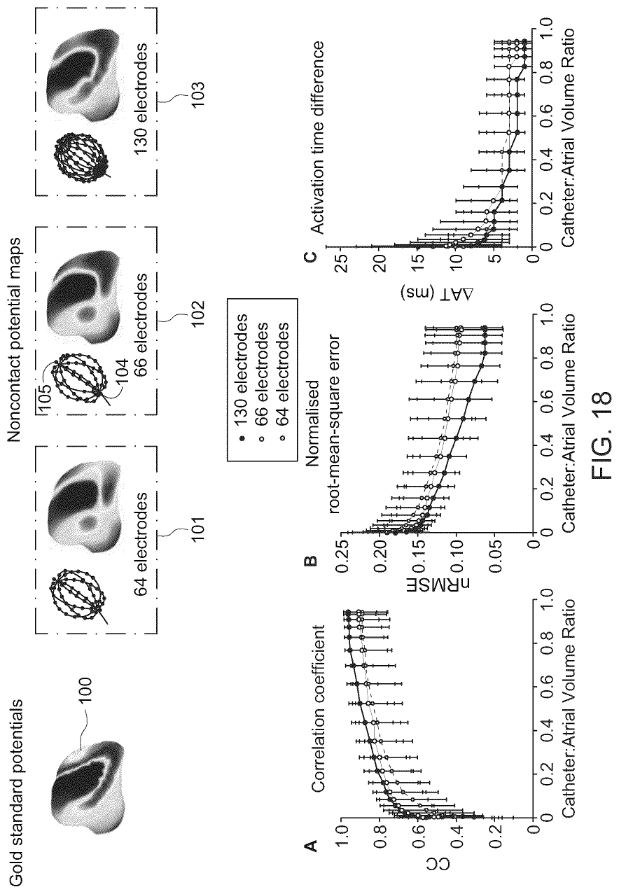

The Constellation™ catheter was intended to be used for contact mapping, and there was no point in locating electrodes at the proximal end (bottom) of the catheter where contact would not occur due to the presence of the guide catheter. However, with non-contact mapping electrodes in this region will record valuable information. Substantial performance benefits of a catheter with just two additional electrodes, 66 in total, is shown in FIG. 18. Non-contact mapping is changing the design constraints for high density mapping catheters.

The electrodes are preferably attached to each spline and use a thin wire running the length of the catheter to connect the electrode to the recording system. Alternatively, the splines may be fabricated using flexible printed circuit board technology, for example, see FIG. 13. This spline 70 in FIG. 13a is relatively easy to manufacture and electrodes may be placed on both sides of the printed circuit board-accommodating a higher number of electrodes for the same physical size of the spline. In the preferred form of this spline of the present invention the width of the spline is 1.4 mm, thickness 0.2 mm and a length suitable to reach the end of the guide catheter. The electrodes are shown as rectangles 71, 72 having dimensions of 2 mm by 0.2 mm. In this example, each of the splines preferably contains six electrodes. However, more electrodes can be placed on the spline as required. Electrodes shown as red rectangles (for example, electrode 71) are those placed on top (one side) of the spline 70 while the blue electrodes (rectangles) (for example, electrode 72) are at the bottom (or other side) of the spline. Non-contact mapping enables electrodes to be located where they will not contact the chamber surface which improves electrode density and could reduce motion artefacts as a chamber surface slides over a contacting electrode.

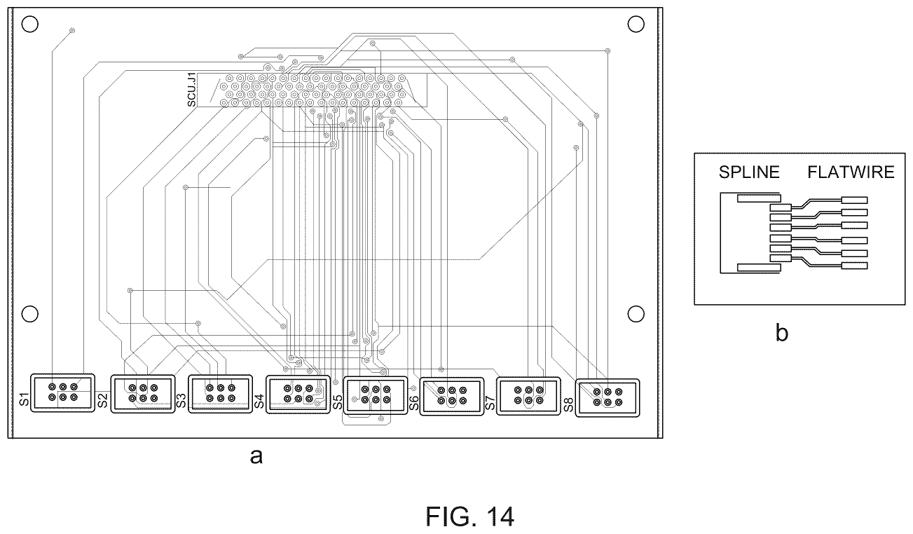

These splines (and indeed the splines of any other of the embodiments of the catheter described herein) can be connected to UnEmap (a University of Auckland electrophysiological high channel count mapping system) through additional connectors and cables. UnEmap provides high quality, multichannel recording of electrical signals. It delivers high spatial electrical mapping with a 448-channel base unit. The printed circuit board (PCB) connecting the splines to UnEmap is shown in FIG. 14. The splines are preferably connected to PCBb in FIG. 14b using a flexible printed circuit board connector. PCBb connects to PCBa in FIG. 14 using a flat ribbon cable then connects to UnEmap using shielded multi-core cables. However, other appropriate connecting mechanisms may be used.

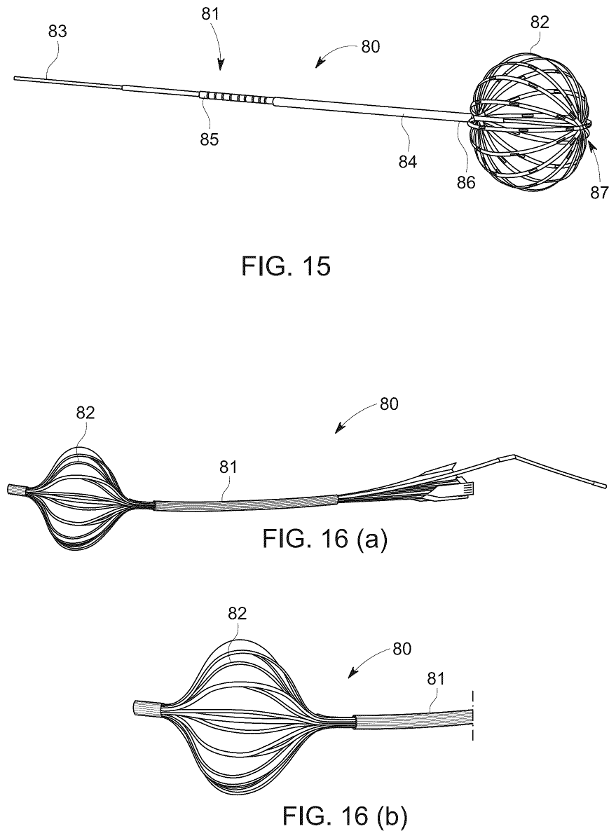

FIG. 15 shows an illustration of the 16-spline catheter of the present invention that supports delivery and extension of the basket once in location. FIGS. 16a and b shows photos of a prototype version of the same catheter 80. FIG. 16a shows the full catheter and FIG. 16b shows a close up of the basket of the catheter. The arm 81 holding the basket 82 includes an inner rod 83 and two outer tubes 84, 85. The inner rod 83 (preferably with 0.9 mm outer diameter) is preferably made of nitinol. A first movable tube 85 extends about the inner rod 83 and the end of the first movable tube 85 is fixed to the proximal end of the splines 85 (bottom of the basket). The distal end of the inner rod is fixed to the distal end of the splines 87 (top of the basket). Preferably the first movable tube has an outer diameter of 1.2 mm. Movement of the inner rod 83 with respect to the first movable tube 85 controls the expansion and contraction of the basket. When the inner rod 83 is extended maximally with respect to the first movable tube 85, the basket is closed and able to be advanced through the second movable tube 84—a guide catheter. The second movable tube 84, preferably with an outer diameter of 3.5 mm, guides the advancement of the first movable tube 85, basket catheter 82 and inner rod 83 to the location inside the heart chamber. When the basket is located inside the chamber, the inner rod 83 is retracted with respect to a stationary first movable tube 85—this action expands the spline to form an open catheter as illustrated. In FIG. 15 the spline connectors are not shown to provide a clearer view of the rod and tubes. The basket catheter in this embodiment has 16 splines with 6 electrodes on each spline.

However, there is likely to be difficulty in manufacture of a 16-spline catheter. As an alternative, a 8 splines catheter with at least 8 electrodes on each spline (with a PCB, the electrodes can be on either side) may provide as good as results. In this form, it is preferred to have even spacing over the surface of the basket catheter, so that results in the spines being different and is likely to result in one polar electrode on one spline servicing an area of the basket without neighbouring splines needing a polar electrode.

### Catheter Testing

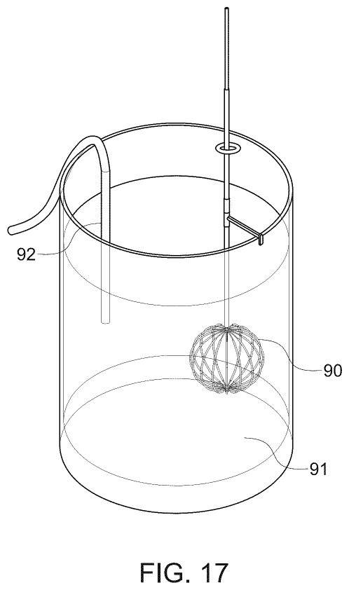

A test right with a saline solution bath was used to check the electrical connectivity of individual electrodes on the splines of the catheter shown in FIGS. 15 and 16 to the UnEmap system. Some elements of the test rig are shown in FIG. 17. The assembled catheter 90 was immersed in a 0.9% sodium chloride solution bath 91. Electrical current was delivered via a wire 92 opposite the catheter 90, attached to a signal generator (Agilent 3320A). The signal used was a sinusoidal pulse with amplitude of 100 mV and width of 6 s. A 5-minute stabilization period was allowed then 5 minutes of recordings. The electrical signals on each electrode were recorded and analysed using UnEmap.

### Electrode Locations

The importance of the electrode locations on the splines of a catheter (any one of the catheters as described above) is shown in this FIG. 18. A gold standard potential map 100 is shown showing an electrical potential distribution over the internal surface of an atrial cavity. The reconstructed non-contact potential maps (to the right) are attempting to re-create the gold standard potential map 100. Three examples of basket design are presented, the first has 64 electrodes 101 in the locations of the commercially available Constellation catheter. The second catheter also has 8 splines but has just two additional electrodes 102, one near each pole of the basket (first electrode 104 at a proximal end of the pole, second electrode 105 at a distal end of the pole), as indicated by the larger dots in the catheter image. The third catheter 103 has 16 splines and increases the number of electrodes to 130.

The performance of the catheter for use in reconstructing the gold standard map will depend on the amount the catheter is expanded to fill the volume of the atrial cavity. The performance is shown using three different metrics as a function of the atrial volume ratio as can be seen in graphs labelled A, B and C. The correlation coefficient is shown in A and is calculated over the whole atrial surface and is seen to always be superior with the 130-electrode catheter compared to the other two catheter designs. When the catheter volume ratio is low, for example less than 0.6, then the importance of electrode placement is easy to see by observing the 66-electrode catheter out-performing the 64-electrode catheter. At a high atrial volume, the 64 and 66-electrode catheters perform in a similar way because when fully extended and in contact with the atrial wall, they are capturing the same information with the same spatial sampling over the majority of the surface. However, at low atrial volume ratio the 66-electrode catheter is performing much better than the 64-electrode catheter and nearly as well as the 130-electrode catheter. As low atrial volume ratio, the spatial distribution of the field available at the catheter has less spatial variability compared to the atrial wall and it sampled adequately by the 66-electrode catheter, so little is gained by the 130 electrodes. However, the 64-electrode catheter is performing worse because of the inferior distribution of the electrodes and the information missing in the polar regions.

These results show how the distribution of electrodes can be evaluated and the quality of the reconstruction map compared to a gold standard map to assess different basket catheter designs. At times different metrics may be useful to assess the clinical utilization of the different catheter designs. The normalized root-mean-square error metric is presented in B. In atrial fibrillation analysis the activation time at different locations is sometimes used to help direct the ablation therapy, and the accuracy of reconstructing activation times is shown in C. These methods are useful in quantifying the performance of different catheter designs and configurations.

These methods support the evaluation of different catheter designs. It is understood that more electrodes are better because they are able to sample an electrical distribution of high spatial complexity.

However, design constraints will limit the number of electrodes that can fit into a delivery guide catheter, and also the reliability and cost of manufacturing the catheter. Given the inverse mapping technique as described enables use of non-contacting electrodes, the design of these catheters and their electrode distribution is not contained by the need to make contact with the chamber surface. This supports locating electrodes where they are able to sample the electrical potentials where there is most spatial complexity.

Unless the context clearly requires otherwise, throughout the description, the words “comprise”, “comprising”, and the like, are to be construed in an inclusive sense as opposed to an exclusive or exhaustive sense, that is to say, in the sense of “including, but not limited to”.

Although this invention has been described by way of example and with reference to possible embodiments thereof, it is to be understood that modifications or improvements may be made thereto without departing from the scope of the invention. The invention may also be said broadly to consist in the parts, elements and features referred to or indicated in the specification of the application, individually or collectively, in any or all combinations of two or more of said parts, elements or features. Furthermore, where reference has been made to specific components or integers of the invention having known equivalents, then such equivalents are herein incorporated as if individually set forth.

Any discussion of the prior art throughout the specification should in no way be considered as an admission that such prior art is widely known or forms part of common general knowledge in the field.

1. A catheter comprising:
an array of splines;
a plurality of electrodes formed at least by electrodes located on each of the splines; and
an arm connected to the array of splines, wherein the arm is configured to move the array of splines from a closed position to an open position forming an open basket, wherein the array of splines can be locked into any one of multiple positions between the closed position and the open position,
wherein in each of the open position, closed position, and the multiple positions between the closed position and the open position, the array of splines form a respective mathematically closed virtual surfaces each with different dimensions, and wherein in each of the mathematically closed virtual surfaces the plurality of electrodes are distributed across the respective closed virtual surface.

2. The catheter of claim 1, wherein some of the splines of the array of splines have more electrodes distributed thereon than other splines of the array of splines.

3. The catheter of claim 1, wherein the electrodes on each spline of the array of splines are spaced along the spline, and the electrode spacing over the array of splines results in some of the splines being different from other splines.

4. The catheter of claim 1, wherein the catheter is configured with different electrode spacing on neighboring splines of the array of splines.

5. The catheter of claim 1, wherein in the open position, the plurality of electrodes are distributed across the array of splines with an inter-electrode spacing, across the respective mathematically closed virtual surface, that is configured to characterize electrical activity within endocardial regions on the order of 10 mm in diameter.

6. The catheter of claim 1, wherein the plurality of electrodes are distributed across the array of splines with the least linear distance between neighboring electrodes.

7. The catheter of claim 1, wherein a first electrode of the plurality of electrodes is located at a proximal pole of the basket, and a second electrode of the plurality of electrodes is located adjacent a distal pole of the basket.

8. The catheter of claim 1, wherein the array of splines comprises an array of eight splines and at least sixty-six electrodes, wherein the at least sixty-six electrodes are spaced evenly over a surface of a basket defined by the array of eight splines when the catheter is advanced to the open position, wherein a first electrode of the sixty-six electrodes is located at a proximal pole of the catheter, and a second electrode of the sixty-six electrodes is located at a distal pole of the catheter.

9. The catheter of claim 8, wherein each of the eight splines comprising the array of eight splines has at least eight electrodes distributed thereon.

10. The catheter of claim 8, wherein each spline of the array of eight splines has a physical arrangement of electrodes along the length of the respective spline, and neighboring splines of the array of eight splines have electrodes distributed with different physical arrangements.

11. The catheter of claim 10, wherein one polar electrode on one spline of the array of eight splines is configured to service an area of the basket without neighboring splines of the array of eight splines needing a polar electrode.

12. The catheter of claim 8, wherein the number of electrodes per spline of the array of eight splines is not equal.

13. The catheter of claim 8, wherein a straight-line distance between neighboring electrodes of the sixty-six electrodes is substantially the same.

14. The catheter of claim 8, wherein each spline of the array of eight splines comprises a top side that faces outwardly of the basket formed by the catheter, and a bottom side that faces inwardly of the basket formed by the catheter, and each spline of the array of eight splines has at least one electrode on the top side and at least one electrode on the bottom side.

15. The catheter of claim 1, wherein when the catheter is advanced to the open position, the catheter comprises:
two additional electrodes, wherein a first electrode of the two additional electrodes is located at the bottom of the open basket, a second electrode of the two additional electrodes is located at the top of the open basket.

16. The catheter of claim 15, wherein the array of splines are joined at the bottom of the open basket and at the top of the open basket.

17. The catheter of claim 15, wherein the catheter has sixty-six electrodes in total, and there are eight splines that comprise the array of splines.

18. The catheter of claim 15, wherein the open basket is configured to substantially fill an atrial chamber of the heart without making contact with the endocardial surface.

19. The catheter of claim 18, wherein at least some of the plurality of electrodes are located where they will not contact the endocardial surface of the atrial chamber.

20. The catheter of claim 15, wherein the catheter is configured to control the resolution of data recorded by the electrodes via expanding and/or contracting the open basket to change the distribution of the plurality of electrodes.

21. The catheter of claim 1, wherein an arrangement of the plurality of electrodes may be altered by withdrawing or advancing the splines into or out of the arm of the catheter.

22. The catheter of claim 1, wherein each spline of the array of splines are adjustable by being withdrawn or advanced out of the arm of the catheter.

23. The catheter of claim 1, wherein the basket is steerable.
